(19) United States
(12) Patent Application Publication
(10) Pub. No.: US 2020/0412145 A1
(43) Pub. Date: Dec. 31, 2020

(54) ULTRA-LIGHT SMALL RECHARGEABLE EMERGENCY POWER BANK AND A MOUNTABLE AIR PURIFIER DEVICE THEREOF

(71) Applicant: SESCOM LTD, Jerusalem (IL)

(72) Inventor: Yehiel ROSENTHALER, Jerusalem (IL)

(21) Appl. No.: 17/017,057

(22) Filed: Sep. 10, 2020

Related U.S. Application Data
(63) Continuation-in-part of application No. PCT/IL2019/050264, filed on Mar. 10, 2019.

(30) Foreign Application Priority Data
Mar. 11, 2018 (IL) 258016

(51) Int. Cl. H02J 7/00; H01T 23/00; H05F 3/02; A61L 9/22

(52) U.S. Cl. CPC H02J 7/0045; H01T 23/00; A61L 9/22; H05F 3/02; H02J 7/0048

(57) ABSTRACT

The present invention relates to a rechargeable emergency card-type power bank for mobile devices, comprising: a battery cell having capacity adapted to recharge a mobile device to enable an emergency operation to be performed; a discharge cable suitable to provide charge from the battery cell to an electrical device; and a recharge port, configured to receive a power cable connector through which the battery cell may be recharged; wherein the power bank comprising reduced size and weight suitable to fit a credit card pocket of a wallet. A further aspect of the present invention relates to a mountable air purifier device that works in tandem with the power bank. The air purifier provides protection to the user from airborne diseases and other particular matter that may be hazardous to the user.

### FIELD OF THE INVENTION

The present invention relates to the field of mobile electric devices. More particularly, the invention relates to a lightweight and slim power bank that is rechargeable and suitable to provide charge to mobile devices. The invention further relates to a mountable air purifier device that works in tandem with the said power bank.

### BACKGROUND OF THE INVENTION

Power banks are used for supplying electrical power to mobile electrical devices (e.g. smartphones, tablets, PDAs, etc.), and provide a solution to the ever-growing demand for high performance alongside the limitation in battery capacity (and therefore battery charge) of mobile electric devices. Accordingly, a charged power bank may be electrically connected to a mobile device and at least partially charge the device.

An additional concern of mobile devices is the mobility of the devices, including their size and weight. It is generally preferred that a mobile device is not larger in size or heavier than the minimal dimensions required for the desirable functionality (e.g. size of display and weight of inner components that enable functionality). Duly, power banks are preferably small in size and weight.

Portable power banks which contain enough charge to recharge a typical mobile electrical device are in many cases relatively large, and are therefore inconvenient to carry around. However, considering that in many cases (e.g. in an emergency situation) a mobile electrical device is only required to be partially charged, it is also unnecessary to have a large power bank.

Several small sized power banks exist, with capacity as low as 1000 mAh. Some of these small power banks are not rechargeable, and are therefore not always practical when a user wishes to reuse the power bank. Furthermore, due to the fact that the capacity of a power bank is derived, among others, from the size of the battery cell of the power bank, in order to reduce the size of a power bank it is required to reduce the charge capacity thereof.

In one aspect, recognizing the need for a power bank designated to provide just enough charge to perform emergency operations on a mobile device, the capacity may be reduced, and therefore so may the size. It would be advantageous to have a portable power bank that is only large enough to contain a battery cell with capacity sufficient to provide enough charge to recharge a mobile device for performing typical emergency operations.

In another aspect, personal protection against airborne diseases has become an increasingly important aspect of everyday life in light of recent pandemic outbreaks. Numerous solutions have become available for individuals to protect themselves from airborne diseases. Amongst them, mask wearing and social distancing have proven effective in slowing the spread of certain airborne pathogens. However, many of these forms of protection are passive and rely on the compliance and behavior of other people, which are not in the control of the individual. Providing the means for an individual to actively protect oneself against airborne diseases, or other particles that may be harmful to the user, has become an essential aspect of personal protection from such diseases.

To address this problem and to protect users from being exposed to potentially harmful airborne diseases while being in contact with other people or in public places, active personal protection against airborne diseases are required. As such, it is desirable to provide a wearable air ionizer that is operatively coupled with the portable power bank to reduce, and at times eliminate, the occurrence of airborne bacterial and viral infections.

It is therefore an object of the present invention to provide a small sized lightweight emergency power bank.

It is another object of the present invention to provide a small sized lightweight emergency power bank that can be recharged.

It is yet another object of the present invention to provide an air purifier device that attaches to the power bank and can be worn by the user in order to actively protect the user from hazardous airborne particles.

Other objects and advantages of the invention will become apparent as the description proceeds.

### SUMMARY OF THE INVENTION

The present invention relates to a rechargeable emergency card-type power bank for mobile devices, comprising:
a. a battery cell having capacity adapted to recharge a mobile device to enable an emergency operation to be performed;
b. a discharge cable suitable to provide charge from the battery cell to an electrical device; and
c. a recharge port, configured to receive a power cable connector through which the battery cell may be recharged;
   comprising reduced size and weight suitable to fit a credit card pocket of a wallet, wherein said power bank has a thickness of less than 4 mm and has a width approximately the width of a credit card.

According to an embodiment of the invention, the capacity of the battery cell is around 500 mAh.

According to an embodiment of the invention, the battery cell is a Li-ion type.

According to an embodiment of the invention, the size of the power bank is between 83.15 and 85.45 mm long, between 53.65 and 53.95 mm wide, and between 3.65 and 3.95 mm thick. For example, the size of the power bank is 83.3 mm long, 53.8 mm, and 3.8 mm thick.

According to an embodiment of the invention, the weight of the power bank is less than 25 grams. For example, the weight of the power bank is 20 grams.

According to an embodiment of the invention, the discharge cable is stored in a groove in a side of the power bank and may be drawn therefrom.

According to an embodiment of the invention, the discharge cable is retractable by using a retractable cord reel that comprises a fixed portion of the discharge cable and an extendable portion of discharge cable, wherein the extendable portion of the discharge cable is wound on a spring-loaded spool and, thus, may be withdrawn or extended from the cord reel and, if desired, retracted back into the cord reel.

According to an embodiment of the invention, the recharge port comprises a micro-USB port suitable to receive a micro-USB connector.

According to an embodiment of the invention, the power bank further comprises a coupling element suitable to couple the power bank to a mobile device.

According to an embodiment of the invention, the power bank further comprises an indicator indicating the current charge rate remaining in the battery cell.

According to an embodiment of the invention, the power bank is combined with an air purifier device that incorporates a negative ion generator to prevent the user from contracting airborne particles that may be deemed hazardous to the user. This combination helps prevent the user from contracting harmful bacteria, viruses, dust, pollen, smoke or any other airborne particles that are in present in the vicinity of the user. In a further embodiment, the user can wear the power-bank/air-purifier combination, on a necklace, other jewelry, or as an attachment to the user's clothing.

### BRIEF DESCRIPTION OF THE DRAWINGS

In the drawings:

FIG. 1A schematically illustrates a front view of a power bank, according to an embodiment of the present invention;

FIG. 1B schematically illustrates a rear view of the power bank of FIG. 1A;

FIG. 1C schematically illustrates a side view of the power bank of FIG. 1A;

FIG. 1D schematically illustrates a top view of the power bank of FIG. 1A; and

FIG. 1E schematically illustrates a retractable cord reel of a discharge cable, according to an embodiment of the invention.

FIG. 2A schematically illustrates a rear view of an air purifier device, according to an embodiment of the present invention;

FIG. 2B schematically illustrates a front view of an air purifier device of FIG. 2A;

FIG. 2C schematically illustrates a side view of an air purifier device of FIG. 2A;

FIG. 2D schematically illustrates a top view of an air purifier device of FIG. 2A;

FIG. 2E schematically illustrates a bottom view of an air purifier device of FIG. 2A;

FIG. 3A schematically illustrates a rear view of an engaged air purifier device of FIG. 2A, fully engaged with the power bank of FIG. 1A;

FIG. 3B schematically illustrates a top view of an engaged air purifier device of FIG. 2A, fully engaged with the power bank of FIG. 1A;

FIG. 3C schematically illustrates a bottom view of an air purifier device of FIG. 2A, fully engaged with the power bank of FIG. 1A;

FIG. 3D schematically illustrates a side view of an air purifier device of FIG. 2A, fully engaged with the power bank of FIG. 1A;

FIG. 4A schematically illustrates a front view of the engaged air purifier device of FIG. 3A provided with a metal neckless, according to an embodiment of the invention; and

FIG. 4B schematically illustrates a side view of the air purifier device of FIG. 2A provided with a metal strip at its rear side, according to an embodiment of the invention.

### DETAILED DESCRIPTION OF THE INVENTION

Reference will now be made to an embodiment of the present invention, examples of which are provided in the accompanying figures for purposes of illustration only. One skilled in the art will readily recognize from the following description that alternative embodiments of the structures and methods exemplified herein may be employed, mutatis mutandis, without departing from the principles of the invention.

The present invention relates to a small sized lightweight rechargeable power bank for battery operated rechargeable electrical mobile devices. The power bank comprises a rechargeable battery cell, i.e. it may be connected by a cable to a power outlet for recharging battery cell in case the charge isn't full. According to an embodiment of the invention, the battery cell is a Li-ion type. The capacity of the battery cell is about 500 mAh, which is lower than other existing power banks, although sufficient to provide enough charge to a mobile device to perform emergency operations, such as make a phone call.

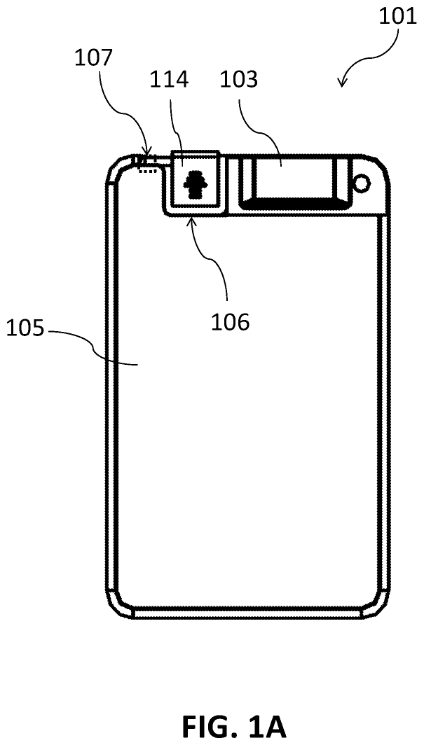

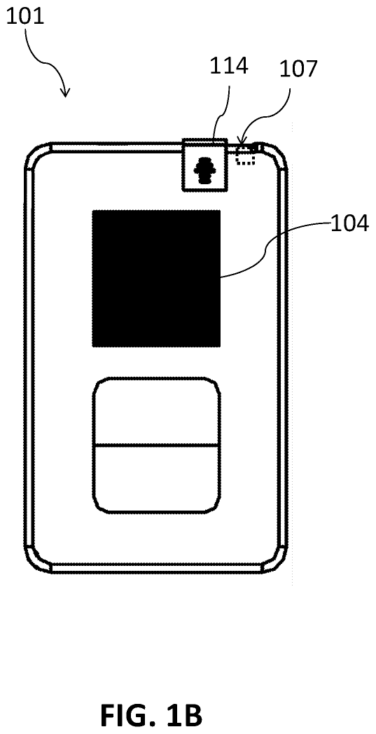

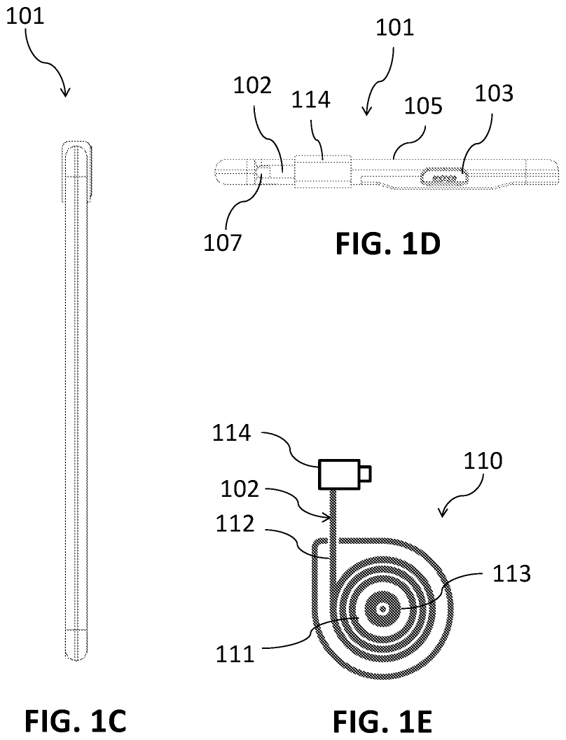

FIGS. 1A-1D show different views of a power bank 101, according to an embodiment of the present invention, where FIG. 1A shows a front view of power bank 101, FIG. 1B shows a rear view of power bank 101, FIG. 1C shows a side view of power bank 101 and FIG. 1D shows a top view of power bank 101. Power bank 101 comprises a discharge cable 102 suitable to provide charge from the power bank 101 to an electrical device connected to discharge cable 102, and a recharge port 103 configured to receive an electric cable that is connected to a power source, through which the battery cell of the power bank 101 is recharged.

According to an embodiment of the invention, discharge cable 102 is stored within housing 105 of power bank 101 and may be drawn therefrom. According to an embodiment of the invention, discharge cable 102 is retractable, e.g., by using a retractable cord reel 110 (as shown in FIG. 1E) that comprises a fixed portion 111 of discharge cable 102 and an extendable portion 112 of discharge cable 102, wherein the extendable portion 112 is wound on a spring-loaded spool 113 and, thus, may be withdrawn or extended from the cord reel and, if desired, retracted back into the cord reel.

Discharge cable 102 comprises a connector 114 located at the termination of extendable portion 112 and having a form suitable to fit the power input port of a corresponding electrical device, such as a lighting connector that is created by Apple Inc. and which is adapted to fit a corresponding electrical device such as an iPhone by Apple Inc.

According to an embodiment of the invention, housing 105 comprises a groove 106 and an opening 107 located adjacent to groove 106 (e.g., both groove 106 and opening 107 located at the power bank's 101 side). Groove 106 adapted to store connector 114 of discharge cable 102 (when the connector is not in use) and opening 107 is used as an output port for discharge cable 102 through which extendable portion 112 of discharge cable 102 is being threaded when pulling out connector 114 out of groove 106 or when discharge cable 102 retracted back.

According to an embodiment of the invention, recharge port 103 is in the form of a micro-USB port suitable to receive a micro-USB connector. According to another embodiment of the present invention, recharge port 103 is configured to receive a power cable connector through which the battery cell may be recharged.

According to an embodiment of the present invention, a coupling element 104 is provided on the rear surface of power bank 101, suitable to couple the power bank to any device, such as a mobile device. For example, the coupling element can be a sort of a sticker, a magnet, etc.

Because the capacity of the battery cell is relatively low (e.g. 500 mAh), the dimensions of power bank 101 may be reduced to those of a credit card. A typical credit card is 85.6 mm long and 53.98 mm wide. The weight of a credit card varies from several grams to 28 grams in metal credit cards. According to an embodiment of the present invention, power bank 101 is between 83.15 and 85.45 mm long, between 53.65 and 53.95 mm wide, and between 3.65 and 3.95 mm thick. According to a specific embodiment of the present invention, power bank 101 is 83.3 mm long, 53.8 mm wide and 3.8 mm thick. Accordingly power bank 101 is suitable to be inserted into a wallet, or any other credit card carrier. According to an embodiment of the present invention the weight of power bank 101 is lower than 25 grams. According to a specific embodiment, power bank 101 weighs 20 grams.

According to an embodiment of the present invention, power bank 101 comprises a charge indicator indicating the current charge rate remaining in the battery cell. The indication may be in the form of one or more LEDs each indicating a different range of charge.

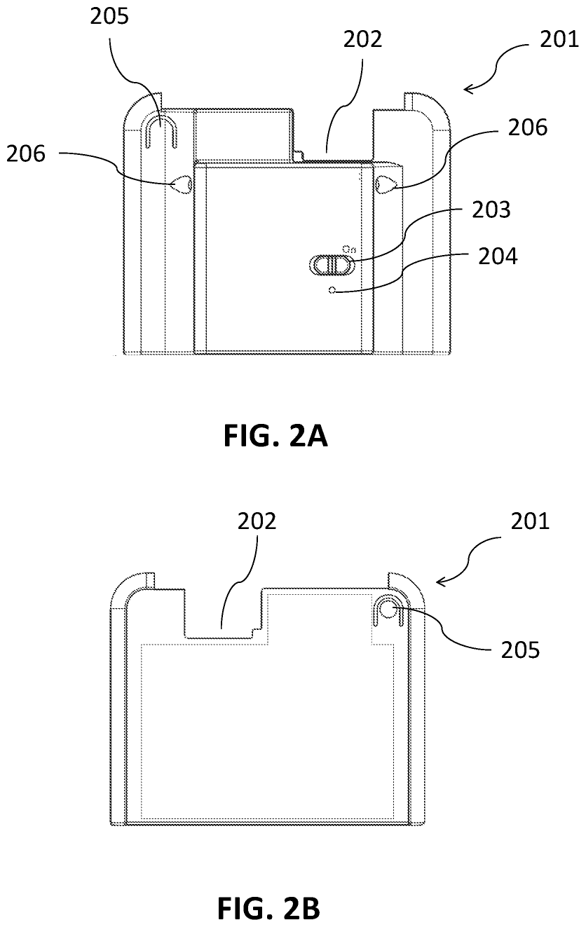

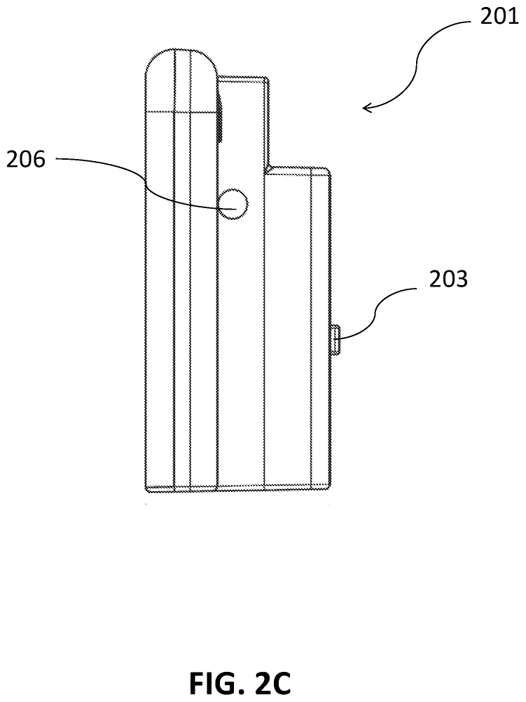

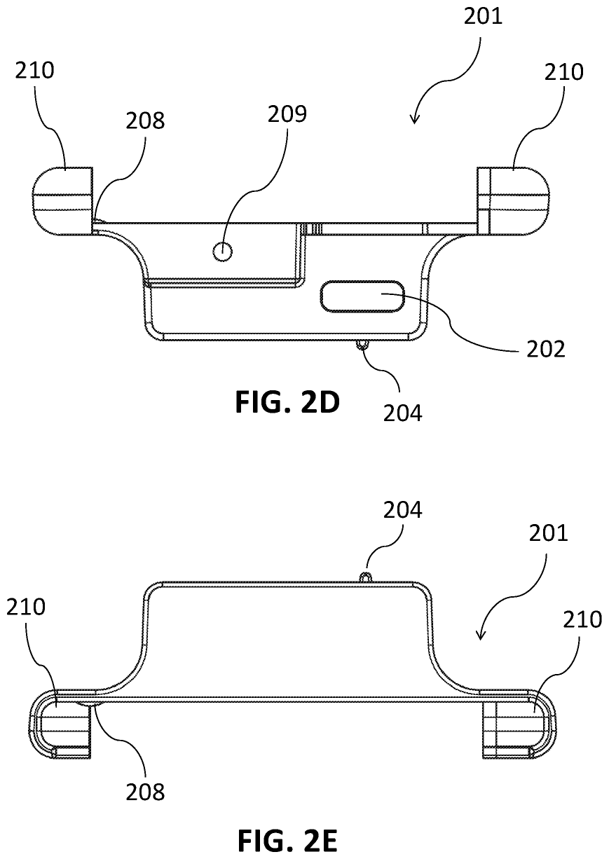

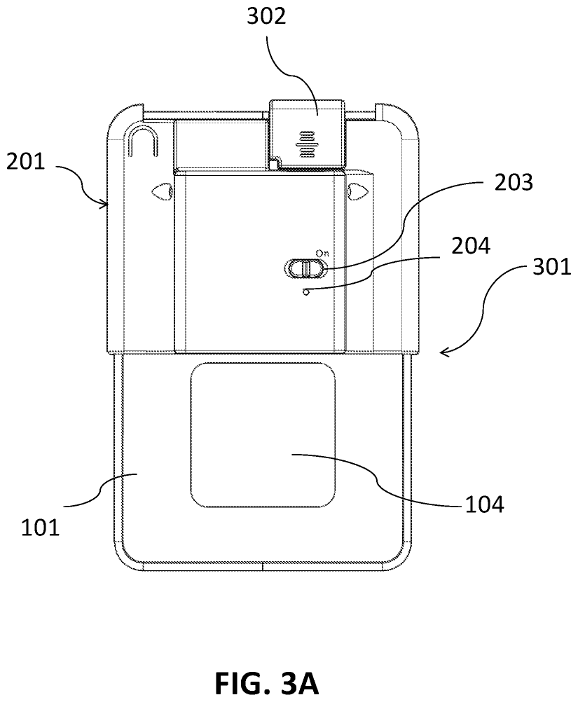

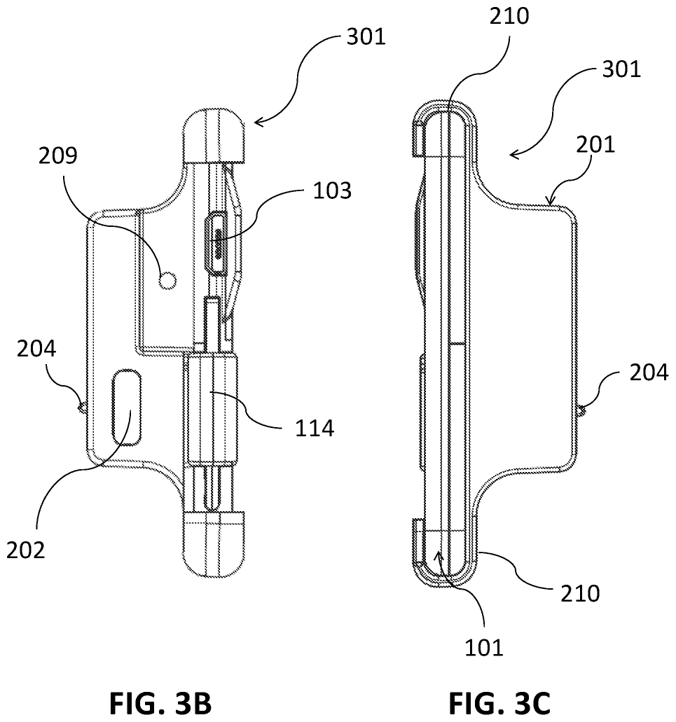

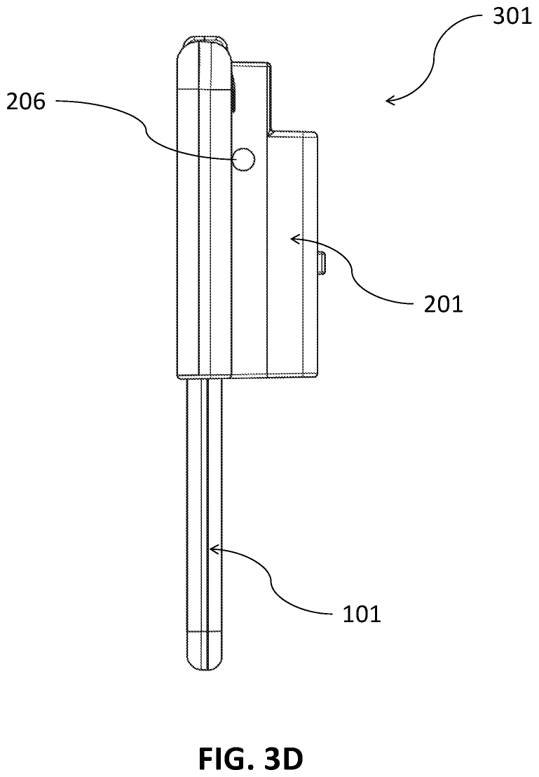

Reference will now be made to a further embodiment of the present invention, examples of which are provided in the accompanying figures for purposes of illustration only. FIGS. 2A-2E show different views of the attachable air purifier device 201 and FIGS. 3A-3D show different views of the engaged device 301 including the power bank 101 and the air purifier device 201.

According to one embodiment the attachable air purifier device 201 can fully engage the power bank 101 without itself having an inbuilt battery. The air purifier and power bank can engage one another, using a reversible locking mechanism, in any number of ways, such as, but not limited to any type of clip, spring-loaded clip, clasp, fastener, opposing magnetic contact and the like. The engaging mechanism is easily reversible such that the power bank can be disconnected to be taken out for easier recharging and/or for removal of the air purifier device. For the purposes of example alone, a protrusion 208 can be used to lock the power bank 101 in place where a corresponding groove on the power bank is located; such a mechanism holds the power bank and the air purifier in place by pressure and/or friction. For the purposes of example alone, a release mechanism (that comprises a clip 205 and protrusion 208) can also be placed on the body of the attachable air purifier to release the hold of the air purifier device 201 with the power bank 101. The body of the attachable air purifier has slots 210 on either side which are shaped to catch the power bank 101 in place and stop the two from disengaging. This arrangement allows for the majority of the power bank 101 to be visible on one side and partly concealed on the opposite side whilst remaining locked in place to the air purifier.

When the power bank 101 is charged, the attachable air purifier device 201 can be mounted for use as an air purifier. The air purifier consists of a negative ion generator whose power is supplied via the power bank and produces negative ions that are ejected in to the atmosphere. For the purpose of clarity, the attachable air purifier and negative ion generator will be referred to interchangeably, since they serve the same function.

In one embodiment a viewing port (not shown) is located on the body of the attachable air purifier to see the indicator light on the power bank.

Once the attachable air purifier device 201 is mounted on to the power bank 101, a power input port 202 receives the connecter 114 from the power bank 101 to be connected via release of the retractable (pull-out) cable 102 which suitably fits the power input port 202 of the negative ion generator. The power input port 202 can be any suitable type of connector such as a type-C connector. In another embodiment, an indicator light 204 turns on the air purifier indicating that the air purifier is supplied with power and is ready for use.

Negative ions are generated using standard methods by electrostatically charged carbon fiber brush generating negatively charged gas ions (e.g. N₂⁻ or O₂⁻). These said negatively charged gas ions are attracted to, and stick to, particulate matter. Air ionisers are routinely used to remove, denature, damage and destroy various types of particulate matter. Airborne particulate matter includes, but is not limited to, dust, pollen, smoke, ash, soot, atmospheric dust, viruses, bacteria, allergens, pathogens, pollen and spores.

Once the power is supplied to the attachable air purifier device 201 the power can be turned on via a switch 203. When operating, the carbon fiber brush generates negative ions which exit through the device and into the atmosphere immediately surrounding it. The negative ion generator may cause particles to stick to the carbon fiber brush, which should be cleaned at regular intervals. Negative ions will cause particulate matter to stick together, increasing the particles' combined weight, causing the particles to fall away from the vicinity of the user. Furthermore, biological airborne particles can be denatured and inactivated by means of negative ions. In one embodiment, negative ions exit the attachable air purifier via an exit port 209; however, there can be multiple exit ports out of which negative ions are released. Furthermore, the negative exit ports 209 can be of any shape. For the purposes of example alone, the exit port 209 can take the shape of a hole (e.g., circular, rectangular, etc.), slit/s or grates, or a combination thereof.

In one embodiment of the invention, the rate at which negative ions can be released from the air purifier can be adjusted by the user. Such a change can be mediated via a dial or switch on the body of the air purifier.

For the purposes of example alone, the attachable air purifier device 201, with a power bank 101 with a battery capacity of 900 mAh which is fully charged, can generate 20 million negative ions in the air and operate for 20 hours continuously. For the purposes of example alone, the attachable air purifier device may use a 5V direct current (DC) with a 250 mA current.

In the full engaged device 301, the connector 114 is fitted into the power input port 202 of the attachable air purifier device 201. The attachable air purifier device 201 can be disconnected at any time in order to connect the power bank 101 with a mobile phone to recharge it.

It should be noted that the attachable air purifier device 201 needs no internal battery, its energy is directly provided by the adjacent power bank 101.

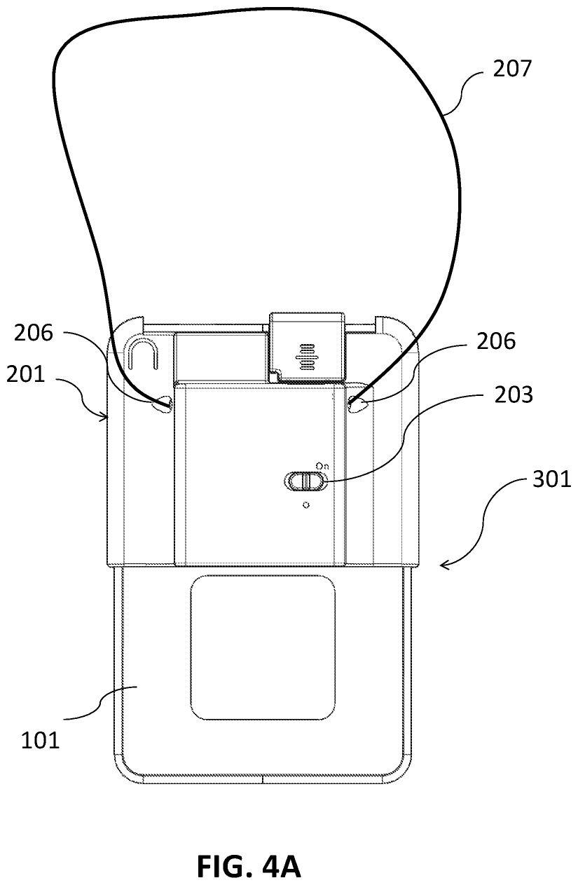

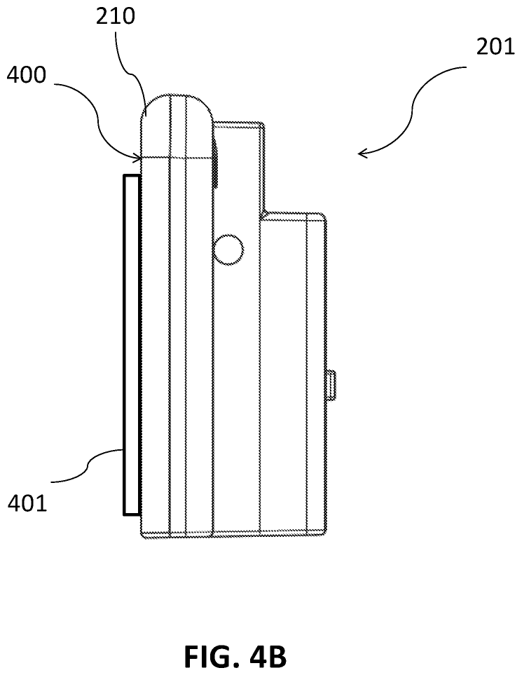

According to an embodiment of the invention, the engaged air purifier device 301 can be worn on a metal necklace 207 (FIG. 4A) which contacts the skin. Holes 206 in the left and right sides of air purifier device 201 can be used to attach metal necklace 207. In this embodiment, the two ends 207a and 207b of the metal necklace 207 pass through holes 206 and connected together. The metal necklace 207 is used to hang the device 301 on the neck of a user so the negative ions can blow towards the face of the user through exit port 209. It is also used to conduct the static electricity to the ground through a wearer's body (i.e., through the body of the user). Referring now to FIG. 4B, according to some embodiments of the invention, the rear portion 400 of the air purifier device 201 comprises a metal strip 401 or other metal element that is adapted to contact the user's body in order to conduct the static electricity to the ground.

According to an embodiment of the invention, the engaged air purifier device 301 can be worn on other forms of jewelry and/or attached to the user's clothing. The engaged purifier device 301 can also be placed in any other location such as a bed side table or a work desk, to further protect the user from contracting airborne particles.

According to an embodiment of the invention, the indicator light 204 can be an LED of any color. According to an embodiment of the invention, the color of the indicator light 204 can indicate the amount of charge remaining in the power bank.

According to an embodiment of the invention, a monitor features on the attachable air purifier device 201 indicating how many minutes of air purifying remains and/or other relevant data pertaining to its functioning.

According to an embodiment of the invention, the attachable air purifier device 201 incorporates an internal fan.

According to an embodiment of the invention, the attachable air purifier device can be used as a stand-alone unit and connected to via any other standard power source.

Although embodiments of the invention have been described by way of illustration, it will be understood that the invention may be carried out with many variations, modifications, and adaptations, without exceeding the scope of the claims.

1. A rechargeable emergency card-type power bank for mobile devices, comprising:
a. a battery cell having capacity adapted to recharge an electrical mobile device to enable an emergency operation to be performed;
b. a discharge cable suitable to provide charge from the battery cell to an electrical device, wherein the discharge cable is retractable by using a retractable cord reel that comprises a fixed portion of the discharge cable and an extendable portion of discharge cable and connector; and
c. a recharge port, configured to receive a power cable connector through which the battery cell may be recharged;
wherein said power having a housing comprising reduced size and weight suitable to fit a credit card pocket of a wallet, wherein said power bank has a thickness of less than 4 mm and has a width approximately the width of a credit card.

2. A rechargeable emergency power bank according to claim 1, wherein the capacity of the battery cell is around 500 mAh.

3. A rechargeable emergency power bank according to claim 1, wherein the battery cell is a Li-ion type.

4. A rechargeable emergency power bank according to claim 1, the size of which is between 83.15 and 85.45 mm long, between 53.65 and 53.95 mm wide, and between 3.65 and 3.95 mm thick.

5. A rechargeable emergency power bank according to claim 4, the size of which is 83.3 mm long, 53.8 mm, and 3.8 mm thick.

6. A rechargeable emergency power bank according to claim 1, the weight of which is lower than 25 grams.

7. A rechargeable emergency power bank according to claim 6, the weight of which is 20 grams.

8. A rechargeable emergency power bank according to claim 1, wherein the discharge cable is stored in a groove in a side of the power bank and may be drawn therefrom.

9. A rechargeable emergency power bank according to claim 1, wherein the extendable portion of the discharge cable is wound on a spring-loaded spool and, thus, may be withdrawn or extended from the cord reel and, if desired, retracted back into the cord reel.

10. A rechargeable emergency power bank according to claim 1, wherein the recharge port comprises a micro-USB port suitable to receive a micro-USB connector.

11. A rechargeable emergency power bank according to claim 1, further comprising a coupling element suitable to couple the power bank to a mobile device.

12. A rechargeable emergency power bank according to claim 1, further comprising an indicator indicating the current charge rate remaining in the battery cell.

13. An air purifier device adapted to be mounted onto the said rechargeable emergency power bank according to claim 1, wherein said air purifier device is a negative ion generator, generating negative ions.

14. The air purifier device according to claim 13, wherein said device locks in place when engaged with said emergency power bank via a reversible locking mechanism.

15. The air purifier device according to claim 13, wherein electrical connection is made to the said rechargeable emergency power bank via the said discharge cable and connector.

16. The air purifier device according to claim 13, wherein said device is adapted be worn on a necklace or on clothing.

17. The air purifier device according to claim 13, wherein the rate of said negative ions can be adjusted.

18. The air purifier device according to claim 13, further comprising at least one metal element that is adapted to conduct static electricity to the ground through a wearer's body.

19. The air purifier device according to claim 18, wherein the at least one metal element is a metal strip attached to a rear side of said device or a metal neckless attached to a body portion of said device.
